(19) United States
(12) Patent Application Publication
(10) Pub. No.: US 2025/0123291 A1
(43) Pub. Date: Apr. 17, 2025

(54) APPLICATION OF HIGH MANNOSE GLYCAN BINDING PROTEINS AND COMPLEX GLYCAN BINDING PROTEINS

(71) Applicant: AGILENT TECHNOLOGIES, INC., Santa Clara, CA (US)

(72) Inventors: Yan Chen, Santa Clara, CA (US); Aled Jones, Santa Clara, CA (US); Oscar Potter, Santa Clara, CA (US)

(21) Appl. No.: 18/887,496

(22) Filed: Sep. 17, 2024

Related U.S. Application Data
(60) Provisional application No. 63/544,168, filed on Oct. 14, 2023.
(60) Provisional application No. 63/590,778, filed on Oct. 16, 2023.

(51) Int. Cl. G01N 33/68; G01N 33/58

(52) U.S. Cl. CPC G01N 33/6854; G01N 33/581; G01N 33/583

(57) ABSTRACT

Methods of quantifying IgG antibodies containing high mannose N-glycans. Methods include immobilizing a protein capable of binding to IgG antibodies containing high mannose N-glycans to a substrate. Methods include adding a sample comprising IgG antibodies containing high mannose N-glycans to the immobilized protein to form a binding mixture. Methods further include adding a high mannose binder protein (HMB)-dye conjugate to the binding mixture such that the HMB-dye conjugate binds to high mannose N-glycans present in the sample. Methods include detecting the IgG antibodies containing high mannose N-glycans bound to the HMB-dye conjugate. Kits for the quantifying IgG antibodies containing high mannose N-glycans are also disclosed.

### CROSS REFERNCE TO RELATED APPLICATIONS

This application claims priority under 35 U.S.C. § 119 (c) to U.S. Provisional Application Ser. No. 63/544,168 titled “APPLICATION OF HIGH MANNOSE GLYCAN BINDING PROTEINS AND COMPLEX GLYCAN BINDING PROTEINS” filed on Oct. 14, 2023, and U.S. Provisional Application Ser. No. 63/590,778 titled “APPLICATION OF HIGH MANNOSE GLYCAN BINDING PROTEINS AND COMPLEX GLYCAN BINDING PROTEINS” filed on Oct. 16, 2023, the contents of each is herein incorporated by reference in its entirety.

### SUMMARY OF THE INVENTION

In accordance with an aspect, there is provided a method of quantifying IgG antibodies containing high mannose N-glycans. The method may include immobilizing a protein capable of binding to IgG antibodies containing high mannose N-glycans to a substrate. The method may include adding a sample comprising IgG antibodies containing high mannose N-glycans to the immobilized protein to form a binding mixture. The method further may include adding a high mannose binder protein (HMB)-dye conjugate to the binding mixture such that the HMB-dye conjugate binds to high mannose N-glycans present in the sample. The method additionally may include detecting the IgG antibodies containing high mannose N-glycans bound to the HMB-dye conjugate.

In some embodiments, the method further may include removing any unbound materials, e.g., impurities, from the binding mixture prior to adding the HMB-dye conjugate.

In further embodiments, the method may include removing unbound HMB-dye conjugate prior to detection of the high mannose N-glycans bound to the HMB-dye conjugate.

In some embodiments, the HMB-dye conjugate further may include an anti-HMB antibody.

In some embodiments, the HMB-dye conjugate further may include a detection enzyme. For example, the detection enzyme may be horseradish peroxidase, β-galactosidase, luciferase, or alkaline phosphatase. In specific embodiments, the detection enzyme may be horseradish peroxidase. In further embodiments, the detection enzyme may include a detection substrate.

In some embodiments, detecting the high mannose N-glycans bound to the HMB-dye conjugate may include detection by an optical measurement. For example, the optical measurement may include absorbance, luminescence, or fluorescence intensity or polarization.

In accordance with an aspect, there is provided a method of quantifying IgG antibodies containing high mannose N-glycans. The method may include indirectly immobilizing a high mannose binder protein (HMB) on a substrate. The method may include contacting a sample including IgG antibodies containing high mannose N-glycans with the immobilized HMB such that any high mannose N-glycans present in the sample bind to the immobilized HMB. The method further may include adding a detection agent comprising one or both of a detection antibody and a dye to the IgG antibodies containing high mannose N-glycans bound to the HMB such that the detection agent binds to the high mannose N-glycans to form an HMB-high mannose N-glycan-detection agent conjugate. The method additionally may include detecting the high mannose N-glycans bound to the HMB-high mannose N-glycan-detection agent conjugate.

In some embodiments, the HMB may be bound to an anti-HMB antibody that is bound to the substrate. In some embodiments, the HMB may include a polyhistidine tag, e.g., His6, that binds to a metal chelating complex present on the substrate.

In some embodiments, the method may include removing any unbound materials, e.g., impurities, from the bound high mannose N-glycans-HMB prior to adding the detection agent. In further embodiments, the method may include removing any unbound detection agent prior to detection of the HMB-high mannose N-glycan-detection agent conjugate.

In some embodiments, the detection antibody may include a detection enzyme. For example, the detection enzyme may be horseradish peroxidase, β-galactosidase, luciferase, or alkaline phosphatase. In specific embodiments, the detection enzyme may be horseradish peroxidase. In further embodiments, the detection enzyme may include a detection substrate.

In some embodiments, detecting the high mannose N-glycans in the HMB-high mannose N-glycan-detection agent conjugate may include detection by an optical measurement. For example, the optical measurement may include absorbance, luminescence, or fluorescence.

In accordance with an aspect, there is provided a kit for the quantification IgG antibodies containing high mannose N-glycans. The kit may include a protein capable of binding to IgG antibodies. The kit may include a high mannose binder protein (HMB). The kit further may include a dye that can bind to the HMB to form an HMB-dye conjugate. The kit additionally may include instructions that provide for the preparation of a substrate having the protein capable of binding to IgG antibodies immobilized and the production of the HMB-dye conjugate.

In some embodiments, the kit further may include a high-mannose N-linked glycoprotein as a calibration standard.

In further embodiments, the kit may include one or more additional consumable components. For example, the kit may include antibodies, respectively with and without high mannose N-glycans, and a well plate or cuvette, e.g., where the sample is measured by the chosen analytical technique.

### BRIEF DESCRIPTION OF THE DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 is a schematic illustration of various embodiments of assays for the quantification of IgG antibodies containing high mannose N-glycans;

FIG. 2 illustrates a method of quantifying IgG antibodies containing high mannose N-glycans, according to an embodiment; and

FIG. 3 illustrates a method of quantifying IgG antibodies containing high mannose N-glycans, according to another embodiment.

### DETAILED DESCRIPTION

This disclosure is not limited in its application to the details of construction and the arrangement of components set forth in the following description or illustrated in the drawings.

The disclosure is capable of other embodiments and of being practiced or of being carried out in various ways. Also, the phraseology and terminology used herein is for the purpose of description and should not be regarded as limiting. The use of “including,” “comprising,” “having,” “containing,” “involving,” and variations thereof herein is meant to encompass the items listed thereafter and equivalents thereof as well as additional items.

Antibodies, including monoclonal IgGs (mAbs), have characteristic N-glycosylation sites in the fragment crystallizable (Fc) region, sometimes referred to as the Asp-297 glycosylation site. The structure of the glycans present at these glycosylayion sites can affect the function of the antibody or indicate useful information about the cells from which it was expressed. An equivalent glycosylation site is also present on some Fc-fusion proteins which, like mAbs, are finding applications as therapeutics.

The aforementioned glycosylation sites are occupied by either “complex type,” “hybrid type,” or “high mannose” type N-glycans. The presence of high mannose N-glycans on IgG molecules reduces the therapeutic efficacy of antibodies that rely on antibody-dependent cell-mediated cytotoxicity (ADCC) for mechanism of action, and increases the rate of serum clearance, making it an “impurity” in therapeutic IgG antibody production. Therefore, control and analysis of the amount of high mannose glycosylation on mAbs is of substantial interest in the pharmaceutical industry. Furthermore, complex type glycans at this site can also affect the function of Fc-containing proteins, modulating the interaction between the protein and the immune system.

Typical approaches for determining which type of glycan is present on a glycoprotein include high resolution mass spectrometry of the glycoprotein, liquid chromatography/mass spectrometry (LC/MS) of protease digests of the glycoprotein, or LC/MS or capillary electrophoresis (CE) of the glycans after enzymatic release from the glycoprotein followed by labeling with a tag that allows for fluorescence detection or which enhances electrospray ionization for mass spectral analysis. High resolution mass spectrometry, e.g., Fourier Transform mass spectrometry, is severely limited by the need for expensive instruments, low sensitivity, and low accuracy due to the presence of multiple mass variants in most proteins. Analysis methods utilizing LC/MS, CE, and optical detection methods are dependent on laborious and time-consuming sample preparation procedures, expensive instruments, large laboratory footprints to house the instruments, and complicated data analysis.

The creation of therapeutic glycoproteins with the desired type of glycan present depends on cell line engineering such that the glycoprotein product is expressed with the desired type of glycans, optimization of cell culture conditions, and trimming and rebuilding the glycans attached to the glycoprotein after expression using carbohydrate active enzymes.

This disclosure is directed to methods utilizing proteins that bind with high affinity to high mannose glycans, hereafter referred to as high mannose binder (“HMB”) proteins. HMBs can be used to quantify an amount of IgG antibodies containing high mannose N-glycans in a sample. The IgG antibodies containing high mannose N-glycans bound to the HMB can be detected using suitable detection schemes, e.g., colorimetric assays or enzyme-linked immunoassay (ELISA) protocols. In addition, the methods and kits disclosed herein can further be applied to complex glycan binding proteins (“CGB”) where the target of analysis or purification is glycoproteins containing complex glycans attached rather than glycoproteins containing high mannose glycans.

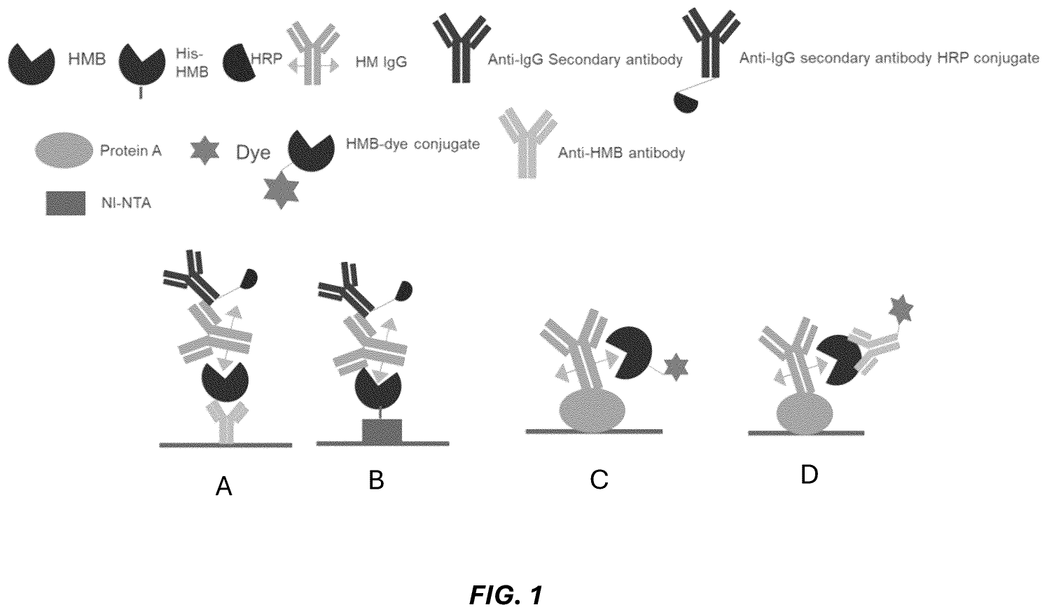

FIG. 1 illustrates schematically the assays of this disclosure used to quantify an amount of IgG antibodies containing high mannose N-glycans in a sample. In FIG. 1, the two assays labeled A and B are termed “sandwich assays” that utilize an indirect linkage between a substrate and HMB and includes a detection agent with one or both of a detection antibody and a dye, optionally with a detection enzyme. As further illustrated in FIG. 1, the two assays labeled C and D are termed “indirect assays” that utilize a binding protein immobilized on a substrate that can bind to IgG antibodies containing high mannose N-glycans. Once the IgG antibodies containing high mannose N-glycans are bound to the binding protein, a label, e.g., HMB conjugated to a dye, can be used to detect the IgG antibodies containing high mannose N-glycans bound to the binding protein.

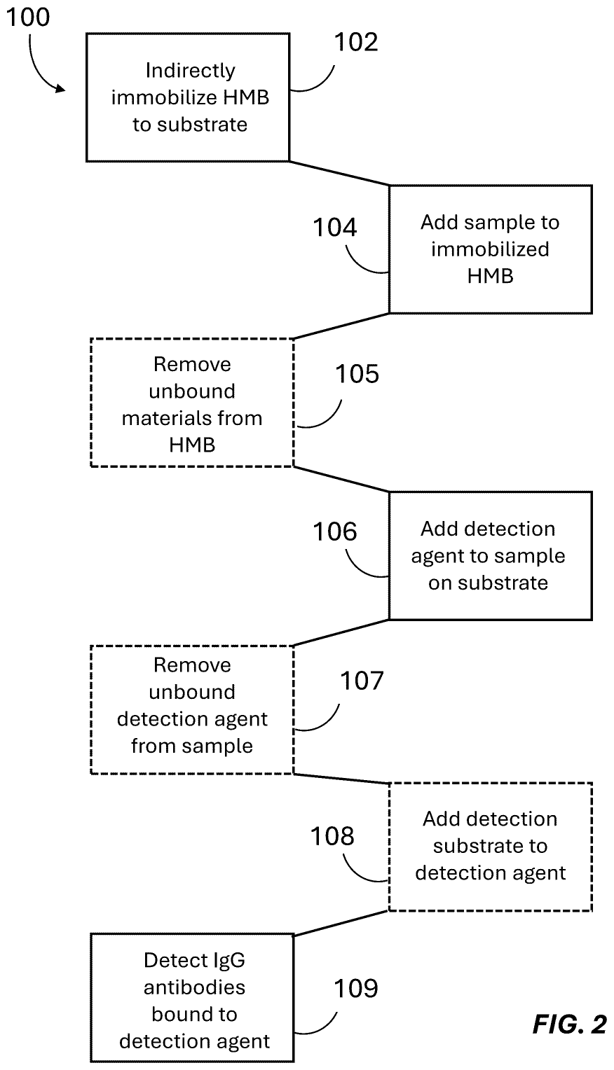

An embodiment of a method of quantifying IgG antibodies containing high mannose N-glycans is illustrated in FIG. 2. In FIG. 2, method 100 includes a first step 102 of indirectly immobilizing a high mannose binder protein (HMB) on a substrate. By “indirect,” it is meant that the HMB in immobilized to the substrate by one or more intermediate molecules, compounds, or the like. As a non-limiting example, HMB can be indirectly immobilized to a substrate by using an anti-HMB antibody, i.e., an antibody that can coordinate with the non-high mannose domains of the IgG antibodies containing high mannose N-glycans, that is immobilized on the substrate. In some cases, HMB can be indirectly immobilized to a substrate by using a compound that is capable of coordinating with the HMB or a modified HMB. As another non-limiting example, metal chelating complexes, e.g., acetylacetone (acac), oxalate, nitrilotriacetic acid (NTA), ethylenediamine (en), and deferoxamine, can coordinate to HMB either natively or through chelation with one or more substituted positions on the HMB. For example, HMB can include an additional polyhistidine tag, e.g., His6, that can bind to the metal chelating complex nickel-nitrilotriacetic acid (Ni-NTA) that is present on the surface of the substrate. Other indirect mechanisms for immobilizing HMB to the substrate are within the scope of this disclosure.

The substrate illustrated in FIG. 2 can be any suitable biocompatible substrate. Some non-limiting examples of substrates include, but is not limited to, well plates made from polystyrene, polycarbonate, glass, quartz, or olefinic polymers, e.g., polypropylene, glass vessels, glass slides, chromatography media, e.g., silica beads, or magnetic beads. In a non-limiting example, magnetic beads can be used as a substrate to immobilize HMB for low throughput quantification of IgG antibodies containing high mannose N-glycans using a commercial plate reader or a cuvette. In another non-limiting example, the HMB can be immobilized onto magnetic beads that can then be used to pull down IgG antibodies containing high mannose N-glycans for purification, cleanup, isolation or as part of a further analytical step or process. As another non-limiting example, the HMB can be immobilized on a chromatography bead, chromatography resin, chromatography matrix, or a monolith to create an affinity column to separate IgG antibodies containing high mannose N-glycans from IgG antibodies that do not contain high mannose N-glycans and other substances. Other substrates for immobilizing HMB to the substrate are within the scope of this disclosure.

With continued reference to FIG. 2, the sample comprising IgG antibodies containing high mannose N-glycans is placed into contact with the immobilized HMB on the substrate at step 104. In this step, the high mannose N-glycans present on the IgG antibodies in the sample bind to the immobilized HMB. In addition to the bound IgG antibodies, the sample includes materials that are unbound, i.e., not coordinated to the HMB or the reagents on the substrate, e.g., impurities or unbound components of the sample. These unbound materials can be removed via a washing step at step 105. Washing the unbound materials, e.g., impurities, away from the bound IgG antibodies containing high mannose N-glycans on the immobilized HMB is performed with any suitable solvent, solution, or buffer using methods known in the art, e.g., magnetic forces, filtering, and centrifugation.

Once the unbound materials, e.g., impurities, are removed from the bound IgG antibodies containing high mannose N-glycans on the immobilized HMB, a detection agent can be added to the IgG antibodies containing high mannose N-glycans bound to the HMB. The detection agent is chosen to bind to the high mannose N-glycans such that an HMB-high mannose N-glycan-detection agent conjugate is formed. The detection agent includes one or both of a detection antibody and a dye that can provide a detectable signal upon interrogation by a suitable detection scheme. In some embodiments, the detection antibody can include a secondary antibody, i.e., an antibody that binds to a primary antibody that is specific to a protein or antigen. As it pertains to this disclosure, a detection antibody can be an anti-IgG secondary antibody that can bind to the bound IgG antibodies containing high mannose N-glycans on the immobilized HMB.

In addition to the detection antibody, the detection agent can, in some embodiments, include a detection enzyme. Detection enzymes are utilized with suitable detection substrates to produce a detectable signal, such as a color change, fluorescence, or chemiluminescence. For example, the detection enzyme may be horseradish peroxidase (HRP), β-galactosidase, luciferase, or alkaline phosphatase. In specific embodiments, the detection enzyme may be horseradish peroxidase. In cases where the detection enzyme is HRP, typical detection substrates include 3,3′,5,5′-tetramethylbenzidine (TMB), 2,2′azino-di-[3-ethylbenzthiazoline-6-sulfonic acid] (ABTS), 3,3′-diaminobenzidine (DAB), and luminol. This disclosure is in no way limited by the detection enzyme and corresponding substrates used to provide a detectable signal indicative of a binding reaction. Once the detection agent is added and any binding reactions have occurred, the sample includes unbound detection agent. The unbound detection agent can be removed via a washing step at step 107 prior to detection of the HMB-high mannose N-glycan-detection agent conjugate. Washing the unbound detection agent is performed with any suitable solvent, solution, or buffer using methods known in the art, e.g., magnetic forces, filtering, and centrifugation.

With continued reference to FIG. 2, after removal of the unbound detection agent from the sample, a detection substrate is added to the bound detection agent at step 108. Without wishing to be bound by any particular theory, for detection agents including detection enzymes, e.g., HRP and the like, detection substrates such as dyes chemically react with the detection enzyme, producing a precipitate that is colored, electron-dense, or luminescent that can be imaged. Following addition of the detection substrate to the detection agent, the high mannose N-glycans bound to the HMB-high mannose N-glycan-detection agent conjugate can be detected at step 109. The detection of the high mannose N-glycans bound to the HMB-high mannose N-glycan-detection agent conjugate is performed using an optical measurement that irradiates the sample using a suitable light source. The choice of optical measurement is a function of the chosen chemistry of the detection agent. For example, the detection may be colorimetric, i.e., absorbance, or emission, e.g., fluorescence or chemiluminescence. Detection can be performed using any suitable instrument, such as a commercial plate reader, microscope, colorimeter, or other spectrometer. This disclosure is in no way limited by the choice of detection method or the optical regime of detection.

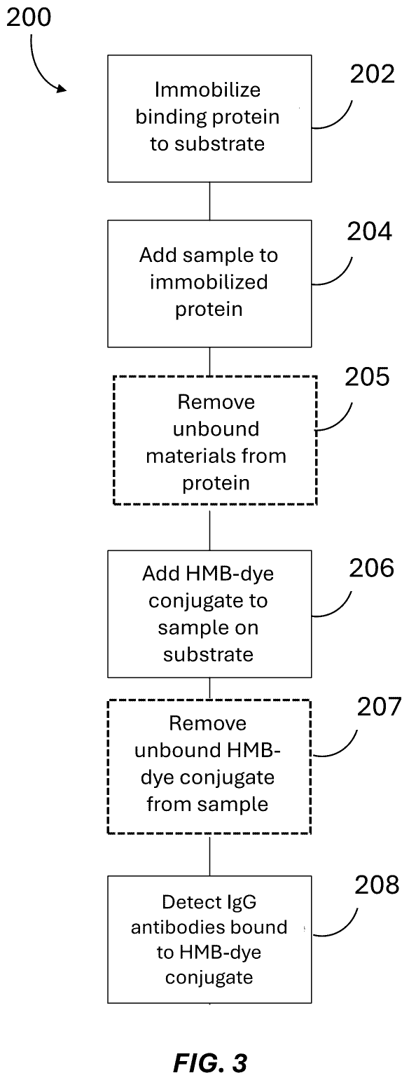

Another embodiment of a method of quantifying IgG antibodies containing high mannose N-glycans is illustrated in FIG. 3. In FIG. 3, method 200 includes a first step 202 of immobilizing a protein capable of binding to IgG antibodies containing high mannose N-glycans to a substrate. There are a number of proteins that are capable of binding to IgG antibodies containing high mannose N-glycans. For example, Protein A, Protein G, Immunoglobulin-binding proteins (IBPs), e.g., secreted immunoglobulin binding protein from group A streptococcus (SibA), IgG Fc-binding protein (Fcybp, a mucin-like secretory Fc receptor protein), Protein disulfide isomerase (PDI), GRP94, and Calnexin can bind to IgG antibodies. In certain embodiments, the protein capable of binding to IgG antibodies containing high mannose N-glycans is Protein A. The substrate illustrated in FIG. 3 can be any suitable biocompatible substrate. Some non-limiting examples of substrates include, but is not limited to, well plates made from polystyrene, polycarbonate, glass, quartz, or olefinic polymers, e.g., polypropylene, glass vessels, glass slides, chromatography media, e.g., silica beads, or magnetic beads. Other substrates for immobilizing a protein capable of binding to IgG antibodies containing high mannose N-glycans to the substrate are within the scope of this disclosure.

With continued reference to FIG. 3, a sample comprising IgG antibodies containing high mannose N-glycans is placed into contact with the protein capable of binding to IgG antibodies containing high mannose N-glycan immobilized on the substrate at step 204. In this step, the high mannose N-glycans present on the IgG antibodies in the sample bind to the immobilized protein to form a binding mixture. In addition to the bound IgG antibodies, the sample includes materials that are unbound, i.e., not coordinated to the protein or the reagents on the substrate, e.g., impurities. These unbound materials, e.g., impurities, can be removed from the binding mixture via a washing step at step 205. Washing the unbound materials, e.g., impurities, away from the bound IgG antibodies containing high mannose N-glycans on the immobilized protein is performed with any suitable solvent, solution, or buffer using methods known in the art, e.g., magnetic forces, filtering, and centrifugation.

Once the unbound materials are removed from the binding mixture, a high mannose binder protein (HMB)-dye conjugate can be added to the binding mixture at step 206. The HMB domain of the HMB-dye conjugate will bind to any IgG antibodies containing high mannose N-glycans on the immobilized protein and the dye will be detectable by any suitable detection scheme. Suitable dyes include, but is not limited to, fluorescein isothiocyanate (FITC), 6-carboxyfluorescein (FAM), ALEXA FLUOR™ 488, DYLIGHT™ 488, cyanine dyes, e.g., Cy3 or Cy5, or rhodamine dyes, e.g., CFR®640. In further embodiments, the HMB-dye conjugate can be used to detect high mannose-containing substances in glycoproteins or cells, i.e., where the sample is not an IgG antibody. This disclosure is in no way limited by the dye used to provide a detectable signal indicative of a binding reaction. Once the HMB-dye conjugate is added and any binding reactions have occurred, the sample includes unbound HMB-dye conjugate. The unbound HMB-dye conjugate can be removed via a washing step at step 207 prior to detection of the IgG antibodies containing high mannose N-glycans bound to the HMB-dye conjugate. Washing the unbound HMB-dye conjugate is performed with any suitable solvent, solution, or buffer using methods known in the art, e.g., magnetic forces, filtering, and centrifugation.

With continued reference to FIG. 3, after removal of the unbound HMB-dye conjugate from the sample, the IgG antibodies containing high mannose N-glycans bound to the HMB-dye conjugate can be detected at step 208. The detection of the IgG antibodies containing high mannose N-glycans bound to the HMB-dye conjugate is performed using an optical measurement that irradiates the sample using a suitable light source. The choice of optical measurement is a function of the chosen chemistry of the dye. For example, the detection may be colorimetric, i.e., absorbance, or emission, e.g., fluorescence or chemiluminescence. Detection can be performed using any suitable instrument, such as a commercial plate reader, microscope, colorimeter, or other spectrometer. This disclosure is in no way limited by the choice of detection method or the optical regime of detection.

In accordance with an aspect, there is provided a kit for the quantification IgG antibodies containing high mannose N-glycans. The kit includes a protein capable of binding to IgG antibodies, e.g., Protein A, Protein G, Immunoglobulin-binding proteins (IBPs), e.g., secreted immunoglobulin binding protein from group A streptococcus (SibA), IgG Fc-binding protein (Fcybp, a mucin-like secretory Fc receptor protein), Protein disulfide isomerase (PDI), GRP94, and Calnexin. The kit includes a high mannose binder protein (HMB). The kit further includes a dye that can bind to the HMB to form an HMB-dye conjugate. The kit additionally includes instructions that provide for the preparation of a substrate having the protein capable of binding to IgG antibodies immobilized and the production of the HMB-dye conjugate.

In some embodiments, the kit further includes a high-mannose N-linked glycoprotein as a calibration standard.

In further embodiments, the kit includes one or more additional consumable components. For example, the kit may include antibodies, respectively with and without high mannose N-glycans, and a well plate or cuvette, e.g., where the sample is measured by the chosen analytical technique.

### EXAMPLES

The function and advantages of these and other embodiments can be better understood from the following examples. These examples are intended to be illustrative in nature and are not considered to be in any way limiting the scope of the invention.

### Prophetic Example 1

In this prophetic example, a protocol for high-mannose glycosylated IgG (“HM IgG”) quantification using an enzyme-linked immunoassay with colorimetric detection is described. HMB can be used to make a colorimetric assay to quantify the amount of Fc HM IgG in a sample using an enzyme-linked immunoassay format.

To prepare this assay, the HMB will be immobilized on a 96-well plate which serves as a solid substrate. The sample containing HM IgG, or HM-IgG standard for the preparation of a calibration curve, will be added to the 96-well plate coated with HMB. HMB can be coated directly on the wells of the 96-well plate or indirectly through an intermediate molecule. An example of such an intermediate molecule that is to be used is an anti-HMB antibody. Another example of an indirect binding system that is to be used is the metal affinity resin nickel nitrilotriacetic acid (Ni-NTA). The Ni-NTA is a metal chelator complex that is to be coated on the plate. The Ni²⁺ atoms in the Ni-NTA can bind to a histidine tag that is part of the HMB structure for immobilization. Upon addition of the sample or the HM IgG standard, binding between the HMB and the high mannose domains of the HM IgG is expected, along with an amount of additional material, e.g., impurities, remaining unbound in the wells of the 96-well plate. The 96-well plate will then be washed with a suitable buffer or solvent to remove any unbound materials, e.g., impurities, from the 96-well plate, leaving behind the bound HM IgG.

Following the washing step, a secondary antibody that is capable of binding to the HM-IgG is to be conjugated with an enzyme label such as horseradish peroxidase, β-galactosidase, luciferase, or alkaline phosphatase, to form a detection agent. Once the detection agent is formed, it is to be added to the 96-well plate with the bound HM IgG. The secondary antibody of the detection agent will bind to the high mannose domains of the HM IgG and any excess detection agent will remain unbound. The excess and unbound detection agent will be washed with a suitable buffer or solvent. The 96-well plate will have a detection substrate added to the wells to produce a chemical reaction with the detection agent indicative of binding. Following this reaction, the 96-well plate will then be analyzed using an optical instrument to measure the optical response of the detection agent. The optical response may be colorimetric (absorbance), fluorescent, or luminescent detection depending on the specific detection agent and detection substrate. Based on the magnitude of the optical response, the quantity of HM-IgG in the sample will be calculated from the calibration curve.

Using horseradish peroxidase as the enzyme label, one example detection substrate is 3,3′,5,5′-tetramethylbenzidine (TMB) which will yield a blue color with absorbance maxima at 370 nm and 652 nm after oxidation. Another example detection substrate for use with horseradish peroxidase is 10-acetyl-3,7-dihydroxyphenoxazine (ADHP) which is a non-fluorescent molecule that will yield a pink color with excitation/emission maxima of 570 nm/585 nm after oxidation.

For the most accurate quantification, a calibration curve made using standard concentrations of HM-IgG is considered necessary. However, for rapid screening or time-dependent incremental monitoring, it may not be necessary to make a calibration curve. The optical response from the sample alone could contain suitable information.

### Prophetic Example 2

In this prophetic example, a protocol for high-mannose glycosylated IgG (“HM IgG”) quantification using a binding protein and a dye is described.

To prepare this assay, the binding protein Protein A will be immobilized on a 96-well plate which serves as a solid substrate. The sample containing HM IgG, or HM-IgG standard for the preparation of a calibration curve, will be added to the 96-well plate coated with Protein A. Upon addition of the sample or the HM IgG standard, binding between Protein A and the high mannose domains of the HM IgG is expected, along with an amount of additional materials, e.g., impurities, remaining unbound in the wells of the 96-well plate. The 96-well plate will then be washed with a suitable buffer or solvent to remove any unbound materials, e.g., impurities, from the 96-well plate, leaving behind the bound HM IgG.

Following the washing step, HMB is to be conjugated with a suitable dye that will provide an optical response upon irradiation. Once the HMB-dye conjugate is formed, it is to be added to the 96-well plate with the bound HM IgG. The HMB in the HMB-dye conjugate will bind to the high mannose domains of the HM IgG and any excess HMB-dye conjugate will remain unbound. The excess and unbound HMB-dye conjugate will be washed with a suitable buffer or solvent. The 96-well plate will then be analyzed using an optical instrument to measure the optical response of the dye. The optical response may be colorimetric (absorbance), fluorescent, or luminescent detection depending on the specific dye conjugated to the HMB. Based on the magnitude of the optical response, the quantity of HM-IgG in the sample will be calculated from the calibration curve.

### Prophetic Example 3

In this prophetic example, other prospective analytical methods that can utilize HMBs binding to molecules containing high mannose N-glycans is described.

It is envisioned that HMBs can be used to make a high mannose containing glycoprotein specific Western Blot. In this aspect, the HMB would be conjugated with a moiety or functional group that allows for direct visualization. In another aspect, the HMB will perform by virtue of a modified antibody/antibodies that bind(s) to the HMB after it has bound to the IgG antibodies containing high mannose N-glycans.

HMBs that bind IgG antibodies containing high mannose N-glycans can also be used in various separation methods as the HMB can be designed to change one or more functional properties of the high mannose N-glycan domain upon binding. For example, the HMB can be designed to cause the high mannose N-glycan domain of the IgG antibody to form a complex with a changed behavior in a separation technique, allowing it to easily separate from the non-HM IgG. Non-limiting examples of this include electrophoretic mobility for capillary zone electrophoresis or gel electrophoresis and the isoelectric point for isoelectric focusing. A further example of the changed behavior in a separation technique upon binding is tailoring the HMB to specific chromatographic methods commonly used in the pharmaceutical industry. Typical chromatographic methods used in the pharmaceutical industry include size exclusion, ion exchange, or affinity separation. The HMB can be designed to change the hydrodynamic volume, charge interaction, or affinity binding of the IgG antibodies containing high mannose N-glycans for each of these chromatographic methods.

As a specific example of modulating affinity binding, the HMB may added to a solution of IgG and incubated for a period of time prior to passing the solution through an antibody binding affinity chromatography column, e.g., a chromatography column containing a medium including with Protein A. The HMB may bind to the IgG antibodies containing high mannose N-glycans and prevent them from interacting with the Protein A, allowing it to pass through the column while the other components, e.g., IgG antibodies that do not contain high mannose N-glycans, are retained for later elution and subsequent processing for analytical or preparative purposes.

In another example, it is envisioned that HMB can be used as a flow cytometry fluorescent label to detect IgG antibodies containing high mannose N-glycans or high mannose containing proteins displayed on cell surfaces.

In another example, it is envisioned that HMB can be used as a cell or tissue imaging indicator by conjugating a colorimetric function as described herein, or through the use of antibodies with a colorimetric function as described herein.

The phraseology and terminology used herein is for the purpose of description and should not be regarded as limiting. As used herein, the term “plurality” refers to two or more items or components. The terms “comprising,” “including,” “carrying,” “having,” “containing,” and “involving,” whether in the written description or the claims and the like, are open-ended terms, i.e., to mean “including but not limited to.” Thus, the use of such terms is meant to encompass the items listed thereafter, and equivalents thereof, as well as additional items. Only the transitional phrases “consisting of” and “consisting essentially of,” are closed or semi-closed transitional phrases, respectively, with respect to the claims. Use of ordinal terms such as “first,” “second,” “third,” and the like in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Having thus described several aspects of at least one embodiment, it is to be appreciated various alterations, modifications, and improvements will readily occur to those skilled in the art. Any feature described in any embodiment may be included in or substituted for any feature of any other embodiment. Such alterations, modifications, and improvements are intended to be part of this disclosure and are intended to be within the scope of the invention. Accordingly, the foregoing description and drawings are by way of example only.

Those skilled in the art should appreciate that the parameters and configurations described herein are exemplary and that actual parameters and/or configurations will depend on the specific application in which the disclosed methods and materials are used. Those skilled in the art should also recognize or be able to ascertain, using no more than routine experimentation, equivalents to the specific embodiments disclosed.

What is claimed is:

1. A method of quantifying IgG antibodies containing high mannose N-glycans, comprising:
immobilizing a protein capable of binding to IgG antibodies containing high mannose N-glycans to a substrate;
adding a sample comprising IgG antibodies containing high mannose N-glycans to the immobilized protein to form a binding mixture;
adding a high mannose binder protein (HMB)-dye conjugate to the binding mixture such that the HMB-dye conjugate binds to high mannose N-glycans present in the sample; and
detecting the IgG antibodies containing high mannose N-glycans bound to the HMB-dye conjugate.

2. The method of claim 1, comprising removing any unbound materials from the binding mixture prior to adding the HMB-dye conjugate.

3. The method of claim 2, further comprising removing unbound HMB-dye conjugate prior to detection of the high mannose N-glycans bound to the HMB-dye conjugate.

4. The method of claim 1, wherein the HMB-dye conjugate further comprises an anti-HMB antibody.

5. The method of claim 1, wherein the HMB-dye conjugate further comprises a detection enzyme.

6. The method of claim 5, wherein the detection enzyme is horseradish peroxidase (HRP).

7. The method of claim 5, wherein the detection enzyme further includes a detection substrate.

8. The method of claim 1, wherein detecting the high mannose N-glycans bound to the HMB-dye conjugate comprises an optical measurement.

9. The method of claim 8, wherein the optical measurement comprises absorbance, chemiluminescence, or fluorescence.

10. A method of quantifying IgG antibodies containing high mannose N-glycans, comprising:
indirectly immobilizing a high mannose binder protein (HMB) on a substrate;
contacting a sample comprising IgG antibodies containing high mannose N-glycans with the immobilized HMB such that any high mannose N-glycans present in the sample bind to the immobilized HMB;
adding a detection agent comprising one or both of a detection antibody and a dye to the IgG antibodies containing high mannose N-glycans bound to the HMB such that the detection agent binds to the high mannose N-glycans to form an HMB-high mannose N-glycan-detection agent conjugate; and
detecting the high mannose N-glycans bound to the HMB-high mannose N-glycan-detection agent conjugate.

11. The method of claim 10, wherein the HMB is bound to an anti-HMB antibody bound to the substrate.

12. The method of claim 10, wherein the HMB comprises a polyhistidine tag that binds to a metal chelating complex present on the substrate.

13. The method of claim 10, comprising removing any unbound materials from the bound high mannose N-glycans-HMB prior to adding the detection agent.

14. The method of claim 13, further comprising removing any unbound detection agent prior to detection of the HMB-high mannose N-glycan-detection agent conjugate.

15. The method of claim 10, wherein the detection antibody comprises a detection enzyme.

16. The method of claim 15, wherein the detection enzyme further includes a detection substrate.

17. The method of claim 15, wherein the detection enzyme is horseradish peroxidase (HRP).

18. The method of claim 10, wherein detecting the high mannose N-glycans in the HMB-high mannose N-glycan-detection agent conjugate comprises an optical measurement.

19. The method of claim 18, wherein the optical measurement comprises absorbance, chemiluminescence, or fluorescence.

20. A kit for the quantification IgG antibodies containing high mannose N-glycans comprising:
a protein capable of binding to IgG antibodies;
a high mannose binder protein (HMB);
a dye that can bind to the HMB to form an HMB-dye conjugate; and
instructions that provide for the preparation of a substrate having the protein capable of binding to IgG antibodies immobilized and the production of the HMB-dye conjugate.
